(19) United States
(12) Patent Application Publication
(10) Pub. No.: US 2026/0193709 A1
(43) Pub. Date: Jul. 9, 2026

(54) METHOD AND SYSTEM FOR INCREASED-ACCURACY IDENTIFICATION OF FETAL GENE DISORDERS IN MATERNAL BLOOD

(71) Applicant: IDENTIFAI GENETICS LTD., Rosh Haayin (IL)

(72) Inventor: Amir BEKER, Rosh Haayin (IL)

(21) Appl. No.: 19/129,667

(22) PCT Filed: Nov. 15, 2023
(86) PCT No.: PCT/IL2023/051183

§ 371 Date: May 13, 2025

(30) Foreign Application Priority Data
Nov. 15, 2022 (IL) 298244

(51) Int. Cl. C12Q 1/6883; G16B 30/10

(52) U.S. Cl. CPC C12Q 1/6883; G16B 30/10; C12Q 2600/156; C12Q 2600/158

(57) ABSTRACT

Disclosed are non-invasive methods for genotyping a fetus, comprising the analysis of sequencing data of maternal cell-free DNA (cfDNA), and parental (maternal and optionally paternal) genomic DNA (gDNA) from a pair parenting the fetus. Using a variant calling approach and assessment of the cfDNA data taken at several time points, accurate genotype predictions are obtained.

### TECHNOLOGICAL FIELD

The present disclosure relates to the field of prenatal genetic analysis.

### REFERENCES

Rabinowitz et al (2019) Genome Res. 29 (3), pp. 428-438
Scotchman et al (2020) Clin. Chem. 66 (1), pp. 53-60
Zhang et al (2019) Nat. Med. 25 (3), p. 439

### BACKGROUND

Non-invasive prenatal testing (NIPT) is the process of assessing the health of an unborn fetus by determining the risk that the fetus will be born with deleterious genetic abnormalities. NIPT relies on the presence of cell-free fetal DNA (cffDNA) as a fraction of total cell-free DNA (cfDNA) circulating in maternal plasma from the early weeks of gestation through to birth.

In an NIPT procedure, blood is drawn from the mother, cfDNA is extracted and sequenced and is then used to gain genetic information about the fetus. Current NIPT procedures are offered in clinics worldwide and can detect large genetic aberrations on a whole-chromosome scale, or very large, specific copy number variations.

NIPT is therefore used for screening chromosomal abnormalities (e.g., trisomies, sub-chromosomal deletions, and duplications), but also for monogenic disorders caused by point mutations. Commercially available next-generation sequencing (NGS) panels consist of only up to 30 genes (Zhang et al (2019)). At the same time, false negative results may occur in tailored tests and panels (Scotchman et al (2020)).

Rabinowitz et al (2019) describe a different approach for genome wide NIPT of monogenic disorders, defining this issue as a unique case of variant calling, termed noninvasive prenatal variant calling. Accordingly, a Bayesian genotyping algorithm utilizes the information of each read, covering each candidate variant, and a machine learning-based fine-tuning step subsequently incorporates information from previously verified results. By accounting for each read, the authors were able to utilize characteristics that separate fetal and maternal DNA, such as fragment length. The algorithm was implemented as Hoobari, the first noninvasive fetal variant caller, that was able to genotype all fetal positions, including biparental loci and indels. However, performance in biparental loci and indels was lower than in positions in which only one parent is heterozygous (WO2021/0340601).

### General Description

In one of its aspects, the present invention provides a method of genotyping a fetus, comprising:
a. receiving sequencing data of maternal cell-free DNA (cfDNA) from the parent carrying the fetus comprising at least two sets of sequencing reads taken at different pregnancy-related time points, wherein one set of sequencing reads being from time point A during pregnancy and at least one other set of sequencing reads being from time point B, wherein time point B being different than time point A and being either before pregnancy or during pregnancy;
b. receiving sequencing data of genomic DNA (gDNA) comprising a third set of sequencing reads of maternal gDNA and optionally a fourth set of sequencing reads of paternal gDNA of the pair-parent to said fetus;
c. analyzing the first and second sets to generate a background reference set;
d. analyzing the second, third, and optionally the fourth, sets to identify sequence reads comprising: (i) a first group of sites at which both parents have two copies of the same allele at a given locus, and (ii) a second group of sites at which at least one of the parents has a variant allele;
e. for each site of the first group, determining a probability that an analyzed cfDNA sequence read originates from said fetus, wherein said determining comprises introducing at least said background reference set to at least one statistical model to obtain a probability value; and
f. using said probability value, classifying each site of the second group as being of either fetal or maternal origin, to thereby genotype said fetus.

In one embodiment, one or both gDNA sequencing data and the cfDNA sequencing data is obtained by a method consisting of whole genome sequencing (WGS), whole exome sequencing (WES), next generation sequencing (NGS), targeted sequencing, panel sequencing, gene sequencing, long-read genome sequencing, paired-end sequencing, single end sequencing, and amplicon sequencing.

In one embodiment, the WGS or WES data is obtained by deep sequencing.

In one embodiment, determining said probability value is based on at least one Sequence Alignment Map (SAM) parameter.

In one embodiment, determining said probability value is based on additional data parameters.

In some embodiments, the additional parameters comprise the length of the reads, GC content, genetic linkage, and haplotypes.

In one embodiment, said step (e) in as defined above further comprises calculating a total fetal fraction.

In one embodiment, the method further comprises constructing a fetal size distribution and a maternal size distribution, wherein determining said probability value comprises binning said fetal size distribution and calculating a fetal fraction for each fragment size bin, and calculating, for at least one size and at least one fragment at said at least one site, a probability that said fragment is fetal, based on a fetal fraction of a respective fragment size bin to which said fragment belongs.

In one embodiment, determining said probability value comprises applying a statistical model, e.g., a Bayesian procedure.

In one embodiment, the method further comprises recalibrating the output of said Bayesian procedure using machine learning.

In one embodiment, the background reference set comprises one or both of: (i) pre-pregnancy maternal cfDNA sequence reads, and (ii) maternal cfDNA sequence reads sampled at one or more time points during the pregnancy.

In one embodiment, the background reference set comprises a time-dependent modulator.

In some embodiments, the time-dependent modulator is based on data parameters comprising the length of the reads, GC content, genetic linkage, and haplotypes.

In one embodiment, the probability values are determined using the Hoobari algorithm.

In one embodiment, the maternal cfDNA is sampled at least at two different time points, such that the reads of cfDNA sequencing data are received from at least one time point before the pregnancy and at one or more time points during said pregnancy with said fetus.

In one embodiment, maternal cfDNA is sampled at two or more different time points during the pregnancy.

In one embodiment, the two or more samples are at time points separated from one another by at least one day.

In one embodiment, the maternal cfDNA is sampled at an early stage of the pregnancy.

In one embodiment, the maternal cfDNA is sampled during the first trimester of the pregnancy.

In another embodiment, the maternal cfDNA is sampled during the second trimester and/or third trimester.

In another aspect, the present invention provides a computer software product, comprising a computer-readable medium in which program instructions are stored, which instructions, when read by a data processor, configure the data processor to (1) receive reads of sequencing data of (i) maternal cell-free DNA (cfDNA) comprising at least two sets of sequencing reads taken at different pregnancy-related time points, and (ii) maternal and optionally paternal genomic DNA (gDNA) from a pair parenting a fetus, and to (2) execute the method of the invention.

In another aspect, the present invention provides a system for genotyping a fetus, comprising: an input utility for receiving reads of sequencing data of (i) maternal cell-free DNA (cfDNA) comprising at least two sets of sequencing reads taken at different pregnancy-related time points, and (ii) maternal and optionally paternal genomic DNA (gDNA) from a pair parenting a fetus; and a data processor configured for analyzing said data for executing the method of the invention.

### BRIEF DESCRIPTION OF THE DRAWINGS

For better understanding the subject matter that is disclosed herein and to exemplify how it may be carried out in practice, embodiments will now be described, by way of non-limiting example only, with reference to the accompanying drawings, in which:

FIG. 1 is a flowchart diagram of a method suitable for fetal genotyping, according to various exemplary embodiments of the present invention.

### DETAILED DESCRIPTION OF EMBODIMENTS

The present invention concerns non-invasive methods for fetal genotyping based on analyses of cfDNA in maternal plasma. The invention is based on the surprising finding that sequence analysis of cfDNA in maternal plasma at several time points, before and/or during pregnancy, generates a genetic “fingerprint”, or profile, that facilitates the determination of the source of the cfDNA with significantly increased accuracy, namely whether it originates from the mother or from the fetus.

According to the invention, at least one of the maternal plasma samples is taken during the pregnancy, preferably at an early stage of the pregnancy, and the at least one more maternal plasma sample is taken either before the pregnancy or at a different time point during the pregnancy. The sequence analysis is performed using deep whole genome sequencing. The multiple analyses of the maternal cfDNA improves accuracy of genotype predictions and enables the identification of various genetic variants in the fetal genome including single nucleotide mutations.

The present invention thus relates to boosting the accuracy of genetic predictions by providing more substantial information on the characteristics of cfDNA in the maternal plasma, thus enabling an improved separation of the fetal cfDNA fraction from total maternal cfDNA.

In an embodiment, the methods of the present invention comprise obtaining maternal cfDNA sequencing data at two or more time points before and/or during pregnancy. Such sequencing data can be obtained from maternal blood samples, including samples that were taken in the past, as a reference of maternal cfDNA signature prior to pregnancy and at earlier stages of pregnancy. An important step in the classification of an identified genetic variant (i.e., a mutation) as being either fetal or maternal is the differentiation process between maternal and fetal sequencing data.

In accordance with the invention, the cfDNA sequencing data obtained at different time points allows the generation of a more accurate profile of maternal cfDNA thus increasing the strength of the difference signal of maternal: fetal cfDNA in statistical models, enabling better probabilistic estimation of the origin of the deep sequencing reads, and consequently, attaining higher accuracy in the predicted genotyping of the fetus.

In accordance with the invention, the incorporation of information on the profile of maternal cfDNA prior to pregnancy and/or the changes that occur in the cfDNA during pregnancy, strengthens the ability to determine whether a specific read is of maternal or fetal origin and thus affirms the fetal genotype predictions.

The cfDNA sequencing data that is obtained during the pregnancy, may be obtained at early stages of the pregnancy, for example at the first trimester, such that if required, intervention procedures may take place. However, under certain circumstances, for example for diagnosing suspected brain morphology anomalies, the cfDNA sequencing data may be obtained also at later stages of the pregnancy, during the second trimester and/or the third trimester. Accordingly, in accordance with the invention, the cfDNA sequencing data may be obtained at any stage during the pregnancy at one or more time points.

In addition to sequencing maternal cfDNA, both paternal and maternal genomic DNA data (gDNA) is also obtained by sequencing DNA from a cell containing sample, using a whole-genome sequencing (WGS) approach, to assign prior probabilities to plasma sequencing reads as to their origins (fetal/maternal).

Generally, the method comprises the following steps:

WGS data collection: gDNA sequencing data from the mother and optionally also the father, as well as cfDNA sequencing data taken from the mother at least at two time points (at least at one time point prior to pregnancy and at least one time point during pregnancy, or at two time points or more, during pregnancy);

### Variant Analysis.

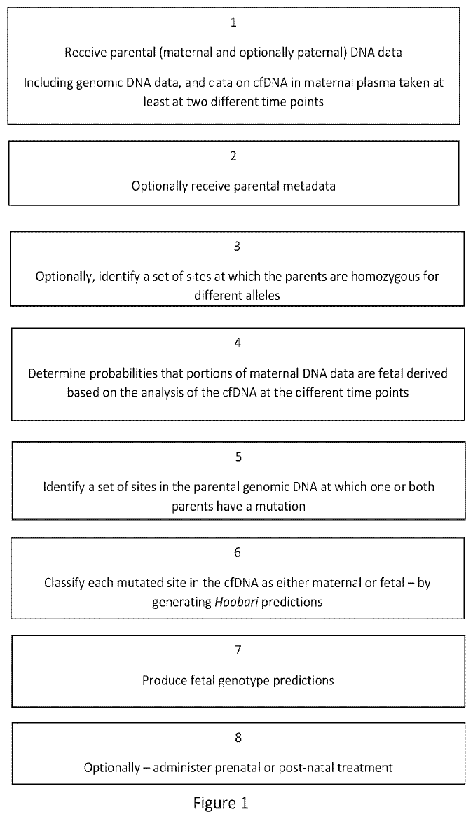

A probability value is assigned to each identified variant, predicting whether the variant is of fetal origin. This probability value is assigned for example, using the algorithm Hoobari, as described in Rabinowitz et al., 2019 and WO2021/340601, and is being calculated for each cfDNA data set received at the different time points to predict whether the variant was indeed inherited by the fetus in every genomic position. A flowchart describing this process is shown in FIG. 1.

Accordingly, in an aspect, the present invention provides a method of genotyping a fetus, comprising:
a. receiving sequencing data of maternal cell-free DNA (cfDNA) from the parent carrying the fetus comprising at least two sets of sequencing reads taken at different pregnancy-related time points, wherein one set of sequencing reads being from time point A during pregnancy and at least one other set of sequencing reads being from time point B, wherein time point B being different than time point A and being either before pregnancy or during pregnancy;
b. receiving sequencing data of genomic DNA (gDNA) comprising a third set of sequencing reads of maternal gDNA and optionally a fourth set of sequencing reads of paternal gDNA of the pair-parent to said fetus;
c. analyzing the first and second sets to generate a background reference set;
d. analyzing the second, third, and optionally the fourth sets to identify sequence reads comprising: (i) a first group of sites at which both parents have two copies of the same allele at a given locus, and (ii) a second group of sites at which at least one of the parents has a variant allele;
e. for each site of the first group, determining a probability that an analyzed cfDNA sequence read originates from said fetus, wherein said determining comprises introducing at least said background reference set to at least one statistical model to obtain a probability value; and
f. using said probability value, classifying each site of the second group as being of either fetal or maternal origin, to thereby genotype said fetus.

“Blood sample” herein refers to a whole blood sample that has not been fractionated or separated into its component parts as well as to a fractionated blood sample. Whole blood is often combined with an anticoagulant such as EDTA or ACD during the collection process but is generally otherwise unprocessed.

“Blood fractionation” is the process of fractionating whole blood or separating it into its component parts. This is typically done by centrifuging the blood. The resulting components are: (a) a clear solution of blood plasma in the upper phase (which can be separated into its own fractions), (b) a buffy coat, which is a thin layer of leukocytes (white blood cells) mixed with platelets in the middle, and (c) erythrocytes (red blood cells) at the bottom of the centrifuge tube in the hematocrit fraction.

“Blood plasma” or “plasma” is the liquid component of blood (the blood component excluding cells). It makes up about 55% of total blood by volume. It is mostly water (93% by volume), and contains dissolved proteins including albumins, immunoglobulins, and fibrinogen, as well as glucose, clotting factors, electrolytes, hormones, carbon dioxide, and cell free DNA.

Blood plasma can be prepared by centrifuging a tube of whole blood in the presence of an anti-coagulant until the blood cells are separated and pulled down to the bottom of the tube. The blood plasma is then poured or drawn off.

“Cell-free DNA” (cfDNA) also referred to as “circulating free DNA” are DNA fragments existing outside of cells in vivo circulating in body fluids such as blood plasma. The fragments of cfDNA typically have lengths ranging from about 150 to 200 base pairs (bp), and averaging about 170 bp, which presumably relates to the length of a DNA stretch wrapped around a nucleosome. During pregnancy, cell-free fetal DNA can be found circulating in maternal plasma. Thus, the cfDNA in maternal plasma is a mixture of both maternal and fetal DNA; both the amount of cfDNA and the fraction of fetal DNA within it increase throughout pregnancy.

The term cfDNA also refers to fragments of DNA that have been obtained from the in vivo extracellular sources and separated, isolated, or otherwise manipulated in vitro. cfDNA can be obtained by extracting DNA from blood plasma after removal of intact cells. Methods for extracting cfDNA are well known in the art, for example, as shown in the Examples below.

The term “genomic DNA” or “gDNA” herein refers to DNA existing in a cell in vivo and containing a complete genome of the cell or organism. The term also refers to DNA that has been obtained from the in vivo cell and separated, isolated, or otherwise manipulated in vitro. Typically, the cell is isolated prior to being subjected to lysis to produce in vitro cellular DNA. The term gDNA as used herein does not include cfDNA.

The term “sample” herein refers to a sample typically derived from a biological fluid, cell, tissue, organ, or organism comprising a nucleic acid or a mixture of nucleic acids comprising at least one nucleic acid sequence that is to be analyzed for the presence of a genetic variant. Such samples include but are not limited to blood or a blood fraction (for example, peripheral blood mononuclear cells) obtained from whole blood samples.

The sample is preferably obtained from a human subject.

The sample may be used directly as obtained from the biological source or following a pretreatment to modify the character of the sample. The sample may be used fresh or thawed after being frozen.

The DNA is extracted using standard protocols, e.g., as described in the examples below.

The maternal gDNA data, maternal cfDNA data, and optionally the paternal gDNA data is obtained by a sequencing method including, but not limited to, deep whole genome sequencing (WGS), whole exome sequencing (WES), next generation sequencing (NGS), targeted sequencing, panel sequencing, gene sequencing, long-read genome sequencing, paired-end sequencing, single end sequencing, and amplicon sequencing.

The term “Next Generation Sequencing” (NGS) herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

Deep sequencing refers to sequencing a genomic region multiple times, sometimes hundreds or even thousands of times. This NGS approach allows researchers to detect rare genetic variants.

As used herein the term “deep whole genome sequencing” refers to deep sequencing of the entire genome. In the context of the present invention cfDNA circulating in maternal blood plasma prior to and/or during pregnancy is subjected to deep whole genome sequencing. The sequencing is repeated multiple times, for example, but not limited to between 5 times (5×) and 2000 times (2000×), e.g., 5 times (5×), 10 times (10×), 20 times (20×), 30 times (30×), 50 times (50×), 100 times (100×), 200 times (200×), 300 times (300×), 500 times (500×), 1000 times (1000×), 2000 times (2000×). In one non-limiting example, the cfDNA in maternal plasma is sequenced 300 times (300×), e.g., as described in the Example below. In addition, maternal and paternal gDNA is also subjected to WGS. Such genomic DNA may be obtained from any cell type, for example from blood cells, e.g., leukocytes. In an embodiment, whole genome sequencing of paternal and maternal gDNA is performed to a targeted depth of between about 20× and 40×, for example 30×.

Whole genome sequencing may be performed using any method known in the art, for example, the HiSeq X Ten System (Illumina), HiSeq 4000 (Illumina), nanopore WGS sequencing using MinION device (Oxford Nanopore Technologies), and WGS by Ultima Genomics.

The sequencing generates “reads” that are sequences of DNA fragments of varying lengths. Typically, though not necessarily, a read represents a short sequence of contiguous base pairs in the sample. The read may be represented symbolically by the base pair sequence (in A T C G). It may be stored in a memory device and processed as appropriate. A read may be obtained directly from a sequencing apparatus or indirectly from stored sequence information.

The sequencing input may be of long reads (e.g., from about 1 KBP (kilogram base pairs) to about 100 KBP, or more) or short reads (e.g., from between about 50 base pairs and 400 base pairs), or a combination of long reads and short reads.

After sequencing, the regions of overlap between reads are used to assemble and align the reads to a reference genome reconstructing the full DNA sequence.

As used herein, the terms “align”, “alignment”, or “aligning” refer to the process of comparing a read to a reference sequence and thereby determining whether the read is contained in the reference sequence. If the reference sequence contains the read, the read may be mapped to a particular location in the reference sequence. In some cases, alignment simply tells whether the read is present or absent in the reference sequence.

Optionally, additional information (also referred to herein as “metadata”) pertaining to one or both the parents is also received. The received metadata optionally and preferably includes at least one, more preferably more than one, of the following features: age, country of origin (/descent), and known genetic conditions.

Sequence alignment techniques that can be used according to some embodiments of the present invention include, without limitation, Burrows Wheeler Aligner (BWA), ABA, ALE, AMAP, anon, BAli-Phy, Base-By-Base, BHAOS/DIALIGN, Bowtie, Bowtie 2, ClustalW, CodonCode Aligner, Comass, DECIPHER, DIALIGN-TX, DIALIGN-T, DNA Alignment, DNA Baser Sequence Assembler, EDNA, FSA, Geneious, Kalign, MAFFT, MARNA, MAVID, MSA, MSAProbs, MULTALIN, Multi-LAGEN, MUSCLE, Opal, Pecan, Phylo, Praline, PicXAA, POA, Probalign, ProbCons, PROMALS3D, PRRN/PRRP, PSAlign, RevTrans, SAGA, SAM, Se-AI, STAR, STAR-Fusion, StatAlign, Stemloc, T-Coffee, UGENE, VectorFriends, and GLProbs.

Exemplary variant callers suitable for the present embodiments include, without limitation, Genome Analysis Toolkit (GATK) and Freebayes. For example, Freebayes can comprise an alignment based on literal sequences of reads aligned to a particular target, not their precise alignment. GATK can comprise: (i) pre-processing; (ii) variant discovery; and (iii) callset refinement. Pre-processing can comprise starting from raw sequence data, e.g., in FASTQ or uBAM format, and producing analysis-ready BAM files; processing can include alignment to a reference genome as well as data cleanup operations to correct for technical biases and make the data suitable for analysis; variant discovery can comprise starting from analysis-ready BAM files and producing a callset in VCF format; processing can involve identifying sites where one or more individuals display possible genomic variation, and applying filtering methods appropriate to the experimental design; callset refinement can comprise starting and ending with a VCF callset; processing can involve using metadata to assess and improve genotyping accuracy, attach additional information and evaluate the overall quality of the callset.

Also contemplated are variant callers such as, but not limited to, Platypus, VarScan, Bowtie analysis, MuTect, and/or SAMtools. For example, Bowtie analysis can comprise implementing the Burrows-Wheeler transform for aligning. MuTect can comprise: (i) pre-processing; (ii) statistical analysis; and (iii) post-processing. Pre-processing can comprise an initial alignment of sequencing reads; statistical analysis can comprise using two Bayesian classifiers, one classifier can detect whether a single-nucleotide polymorphism (SNP) is non-reference at a given site and, for those sites that are found as non-reference, the other classifier can ensure that the normal does not carry the SNP; post-processing can comprise removal of artifacts of sequencing, short read alignments, and hybrid capture. SAMtools can comprise storing, manipulating, and aligning sequencing reads stored as SAM files.

In various exemplary embodiments of the invention the method comprises the determination of the probability, for each variant site of the first set, to be of fetal origin.

As used herein, the term “variant” or “genetic variant” refers to a change (also referred to as a mutation) in the DNA sequence. The method of the invention is suitable for the detection of various types of genetic variants, including, but not limited to single nucleotide polymorphism (SNP), copy number variation (CNV), including insertion/deletion (Indel), and aneuploidy, STR (short tandem repeats) variants, expansion mutations, and de novo mutations.

The term “single nucleotide polymorphism” or “SNP” herein refers to a genomic variant at a single base position in the DNA.

The term “copy number variation” (CNV) herein refers to any structural genome variant in which the amount of a certain genomic sequence is altered either increased or decreased. As such CNV encompasses deletions, insertions, duplications, multiplications, and translocations. CNV also encompasses chromosomal aneuploidies and partial aneuploidies.

The term “aneuploidy” herein refers to an imbalance of genetic material caused by a loss or gain of a whole chromosome, or part of a chromosome.

The term “short tandem repeat variant” or “STR variant” (also known as microsatellites) herein refers to variations in short tandemly repeated (STR) DNA sequences. These STR involve a repetitive unit of 1-6 base pairs, and form series of repetitions with lengths of up to 100 nucleotides.

The term “expansion mutation” herein refers to an increase in the copy number of a repeated unit, commonly a di- or trinucleotide.

The term “de novo mutation” herein refers to germline mutations that newly occurred within one generation. Namely, a new genetic variation that appeared in a fetus while none of the parents carry the mutation.

In an embodiment, the determination of the probability of the variant to be of fetal origin comprises constructing a fetal size distribution and a maternal size distribution, binning said fetal size distribution and calculating a fetal fraction for each fragment size bin, and calculating, for at least one size and at least one fragment at said at least one site, a probability that said fragment is fetal, based on a fetal fraction of a respective fragment size bin to which said fragment belongs.

As used herein the term “fetal fraction” or “FF” refers to the portion of fetal cfDNA, within the total amount of cfDNA in the maternal blood. The portion of fetal cfDNA within maternal blood (the fetal fraction) varies throughout the pregnancy, and between individuals.

In an embodiment, said determining the probabilities comprises applying a Bayesian procedure. Optionally, said Bayesian procedure comprises prior probabilities calculated using sequencing data of at least one of said parents.

In an embodiment, this procedure further comprises recalibration of the output of said Bayesian procedure using machine learning.

In a specific embodiment the determination of the probability, for each variant site, to be of fetal origin is performed as described in Rabinowitz et al., 2019 and WO2021/0340601.

As used herein the term “heterozygous” refers to different versions (alleles) of a genomic locus. The term “homozygous” refers to the presence of the same versions (alleles) of the genomic locus.

The term “locus” is used to refer to the specific location of a nucleic acid sequence or variant on a reference chromosome.

In addition, the cfDNA is analyzed to create a maternal cfDNA “fingerprint”, or profile, namely, to identify unique characteristics of the maternal cfDNA. These characteristics include, but are not limited to, the length of the reads, GC content, genetic linkage, haplotypes, and any other measurable characteristic of the cfDNA.

Next, genotype predictions received for earlier time points are used as additional prior distribution parameters and are inputted to the Bayesian estimation process to further boost the accuracy level of the mother-fetus cfDNA separation.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. Disclosed and described, it is to be understood that this invention is not limited to the specific examples, method steps, and compositions disclosed herein as such method steps and compositions may vary somewhat. It is also to be understood that the terminology used herein is used for the purpose of describing specific embodiments only and not intended to be limiting since the scope of the present invention will be limited only by the appended claims and equivalents thereof.

It must be noted that, as used in this specification and the appended claims, the singular forms “a”, “an”, and “the” include plural referents unless the content clearly dictates otherwise.

Throughout this specification, and the Example and claims which follow, unless the context requires otherwise, the word “comprise” and variations thereof such as “comprises” and “comprising” will be understood to imply the inclusion of a stated integer or step or group of integers or steps but not the exclusion of any other integer or step or group of integers or steps.

### EXAMPLE

### Materials and Methods

### Sample Collection and DNA Extraction

Samples from each family were collected during week 11 of the pregnancy with informed consent. DNA from chorionic villus sampling (CVS) was extracted using the DNA Tissue protocol for the MagNA Pure Compact Nucleic Acid Isolation Kit I-Large Volume (Roche Life Science). Peripheral maternal blood was collected using 2-4 Ethylene-diamine-tetra-acetic acid (EDTA) tubes. Plasma was separated from total blood by centrifugation at 4° C. for 10 minutes at 1600×g. The plasma was then centrifuged again at 16,000×g for 10 minutes at room temperature to remove any residual cells. Extraction of cfDNA was performed using the QIAamp Circulating Nucleic Acid Kit (Qiagen). Removal of excess salts resulting from cfDNA purification was conducted using Agencourt AMPure XP beads (Beckman Coulter, Inc.) at a 2× ratio to cfDNA volume. Pure maternal DNA was extracted from leukocytes in the maternal buffy coats, using a protocol that includes (i) buffy coat separation, and (ii) DNA purification using the Gentra Puregene Blood Kit (Qiagen) according to the manufacturer's instructions. Pure paternal DNA was collected and purified similarly.

### Library Preparation and Sequencing

Library preparation for samples that underwent WGS was performed using the TruSeq DNA PCR-Free Library Prep Kit (Illumina) according to the manufacturer's instructions. This was followed by sequencing using the HiSeq X Ten System (Illumina) with 151-bp paired-end reads.

For samples that underwent whole-exome sequencing (WES), library preparation was performed using the SureSelect V5 Exome Kit (Agilent) according to the manufacturer's instructions. Enrichment was achieved by hybridizing prepared genomic DNA to complementary RNA probe. Sequencing was then performed using HiSeq 4000 (Illumina) with 101-bp paired-end reads.

Cell-free DNA samples were not fragmented during library preparation and were sequenced to a requested coverage of 300×, using HiSeq 4000 (Illumina) with 151-bp paired-end reads.

### Alignment to the Genome

Reads were aligned to the Genome Reference Consortium Human Build 38 (GRCh38/hg38) using Burrows-Wheeler v0.7.834 with default parameters. Duplicate reads, resulting from PCR clonality or optical duplicates, and reads mapping to multiple locations were excluded from downstream analysis.

### Variant Calling of Pure Genomic Sequencing Data

Single-nucleotide substitutions and small insertions and deletions were identified using the GATK HaplotypeCaller software v4.2.4.0 applying default parameters. HaplotypeCaller was first run on the aligned sequencing data of both parents together, then on the aligned data of the CVS sample using the variant sites that were identified in the parental genomes. Reported variants were not filtered, so that all reported SNPs and indels were kept for downstream analysis.

### Pre-Processing of Cell-Free DNA Data

HaplotypeCaller was run on the cfDNA sample only at variant sites that were identified in the parental genomes. Using Hoobari, the allele that was observed by each read, together with the read insert-size, was saved in a separate database.

### Noninvasive Fetal Variant Calling

Hoobari was run using the parental variants and the cfDNA pre-processing results database as input. The output was a standard variant call format (VCF) file. The analysis of the results was held using several software dedicated for VCF manipulation, such as vcflib and vcftools.

### Bayesian Noninvasive Genotyping

At each site of interest, a Bayesian calculation was applied. For each possible fetal genotype:

$$P ( G \vert data ) = \frac{P ( data \vert G ) P ( G )}{{\sum }_{i = 1}^{n} P ( data \vert {G}_{i} ) P ( {G}_{i} )}$$

where G is the fetal genotype and Gᵢ is the i^{th} possible fetal genotype out of n possibilities. For bi-allelic variants, it would be either homozygous for the reference allele (AA), heterozygous (Aa), or homozygous for the alternate allele (aa). P(G) is the prior probability for each genotype and was calculated by Mendelian-inheritance laws. The data variable denotes the reads that cover a site and P(data|G) denotes the likelihood function, which is defined in this Example as a product of the likelihood of each read:

$$P ( data \vert G ) = {\prod }_{j = 1}^{m} P ( {r}_{j} \vert G , {G}_{M} , f ) = {\prod }_{j = 1}^{m} ( P ( {r}_{j} \vert fet ) P ( fet ) + P ( {r}_{j} \vert mat ) P ( mat ) ) .$$

The likelihood of a read rⱼ depends on the fetal genotype and is calculated using the maternal genotype and the fetal fraction. P(rⱼ|fet)P(rⱼ|fet) and P(rⱼ|mat) are the probabilities of a read-observation that supports a certain allele, given that the read is fetal or maternal, respectively. This depends on the tested fetal genotype Gᵢ, the maternal genotype G_{M}, and the observed allele. P(fet)P(fet) and P(mat) are the probabilities of observing a fetal or maternal read based only on the fetal fraction, regardless of the allele that it supports. To utilize the size differences between fetal and maternal fragments, the fetal fraction used for each read was calculated only from reads with the same fragment size. For reads that are not properly paired or have a fragment size of >500, the total fetal fraction is used.

1. A method of genotyping a fetus, comprising:
a. receiving sequencing data of maternal cell-free DNA (cfDNA) from the parent carrying the fetus comprising at least two sets of sequencing reads taken at different pregnancy-related time points, wherein one set of sequencing reads being from time point A during pregnancy and at least one other set of sequencing reads being from time point B, wherein time point B being different than time point A and being either before pregnancy or during pregnancy;
b. receiving sequencing data of genomic DNA (gDNA) comprising a third set of sequencing reads of maternal gDNA and optionally a fourth set of sequencing reads of paternal gDNA of the pair-parent to said fetus;
c. analyzing the first and second sets to generate a background reference set;
d. analyzing the second, third, and optionally the fourth, sets to identify sequence reads comprising: (i) a first group of sites at which both parents have two copies of the same allele at a given locus, and (ii) a second group of sites at which at least one of the parents has a variant allele;
e. for each site of the first group, determining a probability that an analyzed cfDNA sequence read originates from said fetus, wherein said determining comprises introducing at least said background reference set to at least one statistical model to obtain a probability value; and
f. using said probability value, classifying each site of the second group as being of either fetal or maternal origin, to thereby genotype said fetus.

2. The method of claim 1, wherein one or both the gDNA sequencing data and the cfDNA sequencing data is obtained by a method selected from a group consisting of whole genome sequencing (WGS), whole exome sequencing (WES), next generation sequencing (NGS), targeted sequencing, panel sequencing, gene sequencing, long-read genome sequencing, paired-end sequencing, single end sequencing, and amplicon sequencing.

3. The method of claim 2, wherein the WGS or WES data is obtained by deep sequencing.

4. The method of claim 1, wherein determining said probability value is based on at least one Sequence Alignment Map (SAM) parameter.

5. The method of claim 1, wherein determining said probability value is based on additional data parameters.

6. The method of claim 5, wherein the additional parameters comprise the length of the reads, GC content, genetic linkage, and haplotypes.

7. The method of claim 1, wherein said step (e) in claim 1 further comprises calculating a total fetal fraction.

8. The method of claim 7, further comprising constructing a fetal size distribution and a maternal size distribution, wherein determining said probability value comprises binning said fetal size distribution and calculating a fetal fraction for each fragment size bin, and calculating, for at least one size and at least one fragment at said at least one site, a probability that said fragment is fetal, based on a fetal fraction of a respective fragment size bin to which said fragment belongs.

9. The method of claim 1, wherein determining said probability value comprises applying a statistical model, e.g., a Bayesian procedure.

10. The method of claim 9, further comprising recalibrating the output of said Bayesian procedure using machine learning.

11. The method of claim 1, wherein the background reference set comprises one or both of: (i) pre-pregnancy maternal cfDNA sequence reads, and (ii) maternal cfDNA sequence reads sampled at one or more time points during the pregnancy.

12. The method of claim 11, wherein the background reference set comprises a time-dependent modulator.

13. The method of claim 12, wherein the time-dependent modulator is based on data parameters comprising the length of the reads, GC content, genetic linkage, and haplotypes.

14. The method of claim 1, wherein the probability values are determined using the Hoobari algorithm.

15. The method of claim 1, wherein the maternal cfDNA is sampled at least at two different time points, such that the reads of cfDNA sequencing data are received from at least one time point before the pregnancy and at one or more time points during said pregnancy with said fetus.

16. The method of claim 15, wherein said maternal cfDNA is sampled at two or more different time points during the pregnancy.

17. (canceled)

18. The method of claim 1, wherein the maternal cfDNA is sampled at an early stage of the pregnancy.

19. (canceled)

20. (canceled)

21. A computer software product, comprising a computer-readable medium in which program instructions are stored, which instructions, when read by a data processor, configure the data processor to (1) receive reads of sequencing data of (i) maternal cell-free DNA (cfDNA) comprising at least two sets of sequencing reads taken at different pregnancy-related time points, and (ii) maternal and optionally paternal genomic DNA (gDNA) from a pair parenting a fetus, and to (2) execute the method according to claim 1.

22. A system for genotyping a fetus, comprising: an input utility for receiving reads of sequencing data of (i) maternal cell-free DNA (cfDNA) comprising at least two sets of sequencing reads taken at different pregnancy-related time points, and (ii) maternal and optionally paternal genomic DNA (gDNA) from a pair parenting a fetus; and a data processor configured for analyzing said data for executing the method according to claim 1.
